SEQUENCE LISTING (8 sequences)

SEQ ID NO: 1 - DNA, 19 bp, Artificial Sequence
  Synthetic
cgcacaaggtgtcttccag

SEQ ID NO: 2 - DNA, 18 bp, Artificial Sequence
  Synthetic
aggatcagtggcgtcgtg

SEQ ID NO: 3 - DNA, 20 bp, Artificial Sequence
  Synthetic
tggtggcagaactgatcaac

SEQ ID NO: 4 - DNA, 20 bp, Artificial Sequence
  Synthetic
ctgcccagtgaagagcagat

SEQ ID NO: 5 - DNA, 18 bp, Artificial Sequence
  Synthetic
tggacgccctcagaaaag

SEQ ID NO: 6 - DNA, 18 bp, Artificial Sequence
  Synthetic
gctccgcaggtgtctcac

SEQ ID NO: 7 - DNA, 19 bp, Artificial Sequence
  Synthetic
tggagaaggccgacatcct

SEQ ID NO: 8 - DNA, 17 bp, Artificial Sequence
  Synthetic
ggcgacgaaggctttgc

(19) United States
(12) Patent Application Publication
(10) Pub. No.: US 2011/0059114 A1
(43) Pub. Date: Mar. 10, 2011

(54) Compositions and Methods for the Treatment of Radioresistant Glioma Stem Cells

(75) Inventors: Bruce A. Sullenger, Chapel Hill, NC (US); Jialiang Wang, Nashville, TN (US); Jeremy N. Rich, Shaker Heights, OH (US)

(73) Assignee: DUKE UNIVERSITY, Durham, NC (US)

(21) Appl. No.: 12/851,482

(22) Filed: Aug. 5, 2010

Related U.S. Application Data
(60) Provisional application No. 61/231,425, filed on Aug. 5, 2009.
(60) Provisional application No. 61/260,054, filed on Nov. 11, 2009.

(51) Int. Cl. A61K 39/395; A61K 31/216; A61K 31/713; A61K 31/27; A61P 35/00

(52) U.S. Cl. 424/174.1; 514/542; 514/44 A; 514/479

(57) ABSTRACT

Compositions and uses of gamma secretase inhibitors (GSIs) for the treatment and inhibition of gliomas in subjects in need thereof are provided. Methods for administering gamma secretase inhibitors in association with radiation for the treatment of radiation resistant gliomas are described. Compositions for the inhibition of Notch signaling and activity are provided.

This application claims the priority benefit of U.S. provisional patent applications, Ser. Nos. 61/231,425 filed Aug. 5, 2009, and 61/260,054, filed Nov. 11, 2009, the entirety of which are herein incorporated by reference.

This disclosure was produced in part using funds from the Federal Government under NIH grant nos.: CA129190, NS047409, NS054276, CA129958, and CA116659. Accordingly, the Federal government has certain rights in this disclosure.

### FIELD OF THE INVENTION

The present disclosure relates generally to fields of cancer and cancer therapy. Specifically, the present disclosure relates to pharmaceutical compositions and methods comprising gamma (γ) secretase inhibitors (GSIs) for the treatment of gliomas and glioblastoma.

### BACKGROUND OF THE INVENTION

Malignant gliomas, including anaplastic astrocytoma (World Health Organization (WHO) grade III) and glioblastoma multiforme (WHO grade IV) are among the most devastating malignancies. Despite recent advances in therapy, treatment of malignant gliomas remains palliative. Median post-diagnosis survival for anaplastic astrocytoma is less than 3 years and for glioblastoma multiforme is 12-14 months [1,2,3]. Maximal surgical resection of the tumor mass followed by radiotherapy and chemotherapy is the standard of care. Gliomas often respond to radiotherapy, however, subsequent recurrence is almost inevitable, suggesting insufficient killing of tumorigenic cells [4]. Recently, cancer cells with stem cell-like properties have been described in a wide range of human tumors. The cancer stem cells model suggests a hierarchical organization of tumors such that a subpopulation of tumor cells at the apex drives and maintains human tumors [5]. Cancer stem cells of glioma and other neoplasms of human central nervous system can be prospectively enriched by selection of the CD133 (prominin-1) cell surface marker [6,7,8,9,10,11], although some tumors may not express CD133 and hence CD133-negative cells may still have characteristics of cancer stem cells [12,13]. Despite expression of certain neural and other stem cell markers (such as CD133, Musashi-1, Nestin, Sox2 and Olig2) by brain tumor stem cells, the definition of cancer stem cells remains functional, requiring sustained self renewal and tumor propagation. The inventors have previously demonstrated that the glioma stem cells were more resistant to radiation compared with the matched non-stem glioma cells due to preferential activation of the DNA damage response pathway [7]. Other groups also reported that the cancer stem cells of breast cancers were relatively resistant to radiation, potentially due to lower levels of reactive oxygen species found in cancer stem cells [14].

The emerging role of cancer stem cells in tumors in response to radiotherapy urges investigation on molecular mechanisms underlying radioresistance of these cells. One of these candidates is the Notch signaling pathway. Notch mediates short-range cellular communication through interaction with ligands presented on neighboring cells [15]. The instrumental roles of Notch in regulation of self renewal and cell fate determination in normal stem cells have been well established [16]. In mammals, Notch signals through four Notch receptors (Notch 1-4) and five ligands (Jagged-1, -2, and Delta-like-1, -3, and -4), which are all type I transmembrane proteins [17]. Activation of Notch involves sequential proteolytic cleavages that eventually lead to release and nuclear translocation of the intracellular domains of Notch receptors (NICDs), and subsequent activation of Notch-dependent transcription. The γ-secretase complex, which mediates the last proteolytic step for release of the NICDs, is essentially required for Notch activation [18,19]. Inhibitors of γ-secretase (GSIs) have been used to block Notch signaling in vitro and in vivo.

The Notch signaling pathway is essentially involved in the maintenance of a variety of adult stem cells, such as breast [20,21], intestinal [22,23] and neural stem cells [24,25,26], through promoting self-renewal and repressing differentiation. Aberrant Notch activity is found in a wide range of human tumors, such as leukemia, breast cancer, and glioma [27,28,29,30]. Taking into account the instrumental functions of Notch in both stem cells and cancer biology, it is not surprising to find that Notch plays an important role in cancer stem cells [31]. Notch inhibition impairs the self-renewal capacity of mammosphere initiating cells derived from ductal carcinoma in situ [21], and reduces colony formation by leukemic stem cells [33]. Activation of Notch through expression of NICD1 promotes growth and neurosphere formation of the SHG-44 glioma cell line [34]. Notch inhibition by a γ-secretase inhibitor (GSI-18) induces apoptosis and differentiation in CD133+ cells enriched from medulloblastoma cell lines, and impairs the tumorigenic capacity of these cells. Of particular interest, apoptosis induced by GSI-18 is 10 fold higher in the primitive cells expressing the neural stem cell marker nestin in comparison to nestin-negative cells, suggesting a preferential Notch dependence of cancer stem cells [35]. It was recently reported that Notch pathway was transiently activated in endothelial cells following radiation, as evidenced by upregulation of Notch pathway components, Jag1 and Hey1 [42].

As for current glioma therapy, Temozolomide, an oral methylating chemotherapeutic agent, became standard of care for newly diagnosed glioblastoma when used concurrently with external beam radiation followed by adjuvant therapy [3]. Despite the benefit of temozolomide, glioblastomas continue to be highly resistant to radiation [4]. Studies provided herein have demonstrated that the cancer stem cell fraction of gliomas was more resistant to radiation than non-stem glioma cells [7]. Given the high tumorigenic capacity of glioma stem cells, this paradigm suggests that disrupting radioresistance of glioma stem cells may augment the efficacy of radiotherapy. Studies have already shown that inhibitors of the checkpoint kinases, Chk1/2, sensitized glioma stem cells to radiation [7]. However, DNA damage checkpoint inhibitors may have limited therapeutic index due to shared dependence of normal cells on these molecules in radioresponse [62].

Thus, there is a need in the art to develop methods and reagents for effectively treating radiation resistant gliomas. Fundamental improvements in brain cancer treatment will require the development of new therapeutic paradigms.

### BRIEF SUMMARY OF THE INVENTION

The invention provides pharmaceutical formulations and methods for treating glioblastomas, including radiation resistant gliomas, and glioma stem cells. The pharmaceutical formulations and compositions of the invention comprise gamma secretase inhibitors (GSIs), and Notch signaling inhibitors. Gamma secretase inhibitors (GSIs) include but are not limited to DAPT, and L685,458. Notch signaling inhibitors include but are not limited to Notch 1(2) antisense RNA, shRNA, siRNA, antibody, dominant-negative form of Notch 1 or Notch 2. In a preferred embodiment, the pharmaceutical compositions are administered in association with radiation therapy.

The capacity of GSIs to increase glioma and glioblastoma sensitivity to radiation treatment is illustrated by results, set forth herein. The present studies demonstrate a radioprotective role of Notch signaling in glioma stem cells. The results show that Notch inhibition by GSIs or Notch1/2-specific shRNA render glioma stem cells more sensitive to radiation. In addition, activation of Notch via expression of Notch 1(2) intracellular domain (NICD1 or NICD2) promotes radioresistance of glioma stem cells. Collectively, the pharmacological and genetic approaches described herein show that Notch activity is critically implicated in the radioresistance of glioma stem cells. These data demonstrate that the Notch pathway is a therapeutic target for improvement of radiotherapy against gliomas and glioblastoma.

The invention provides methods for modulating Notch expression and signaling activity. More specifically, the invention provides method for inhibiting Notch signaling activity via the administration of gamma secretase inhibitors or Notch signaling inhibitors. The invention provides methods for blocking Notch signaling activity as means for increasing glioma cell or glioblastoma sensitivity to radiation.

The invention provides methods for administering pharmaceutical compounds for treating glioma and glioblastoma. The pharmaceutical formulations utilized include gamma secretase inhibitors or Notch signaling inhibitors, examples of which are also provided.

The invention provides methods for using GSIs and Notch signaling inhibitors to treat glioblastoma, and glioma. The invention further provides methods for administering a therapeutically effective amount of a pharmaceutical composition comprising a GSI, Notch signaling inhibitor or combination thereof to a subject suffering from glioblastoma. In a preferred embodiments of the invention, the GSI is DAFT or L685,458.

In one aspect of the invention, the method comprises administering to a subject in need of such treatment an effective amount of a GSI, Notch signaling inhibitor combinations thereof in combination with the administration of a therapeutic effective amount of radiation. In certain embodiments, GSIs and/or Notch signaling inhibitors are administered at a minimal dosage effective for treatment or a maximal dosage under toxicity.

The invention thus provides advantageous alternatives to current methods and pharmaceutical compositions for treating radiation resistant glioma and glioblastoma.

Specific preferred embodiments of the present invention will be better understood from the following more detailed description of certain preferred embodiments and the claims.

### DESCRIPTION OF THE DRAWINGS

These and other objects and features of this invention will be better understood from the following detailed description taken in conjunction with the drawings wherein:

FIG. 1 is a series of graphs illustrating that γ-secretase inhibitors reduce cell growth and clonogenic survival of glioma stem cells following radiation. (A) T4302 CD133+ glioma stem cells were transfected with a Notch-responsive luciferase reporter (RBP-JK reporter) pre-mixed with the vector directing constitutive expression of Renilla luciferase. Cells were left unirradiated or irradiated at 3 Gy, and were then collected to measure luciferase activities at time points as indicated. In this and subsequent luciferase assays, firefly luciferase activity was normalized to the internal control Renilla luciferase activity and the normalized firefly luciferase activity from the sham-irradiated control group was assigned a value of 1. Data are presented as the mean±standard error, (B) T4302 CD 133+ glioma stem cells were left unirradiated or irradiated at 3 Gy. The relative mRNA levels of Hes2 were determined by quantitative real-time PCR at the time points as indicated. (C) T4302 CD 133+ cells were transfected with the RBP-JK reporter and treated with DMSO (vehicle), 2 μM DAPT or 0.5 μM L685,458 over night, and were then collected for luciferase assays. (D) T4302 CD133+ cells were pre-treated with 2 μM DAPT or 0.5 μM L685,458 for 4 hours and irradiated as indicated. Total viable cell numbers were then determined by the CellTiter-Glo Luminescent Cell Viability Assay (Promega) for the following five days. The averaged cell titers on day 1 of the control groups were assigned a value of 1. (E) T4302 CD133+ cells were pre-treated and irradiated as described in FIG. 1D. Cells were then plated to form neurospheres, and the surviving fractions were determined as described in methods. The mean±standard error was derived from 8 replicates within a representative experiment. (F,G) Neurosphere formation of T3359 and T4105 CD 133+ cells treated with GSIs alone, 3-Gy radiation alone, or the combination was determined as described in FIG. 1E. *: p<0.05 by Student's t-test; #: p<0.001 by one-way ANOVA.

FIG. 2 is a series of graphs illustrating that γ-secretase inhibitors enhance radiation-induced cell death in glioma stem cells. (A) T4302 CD 133+ cells were pre-treated with DAPT or L685,458 for 4 hours and irradiated at 3 Gy. Percentage of dead cells was determined by trypan blue exclusion assay at 48 hours after radiation exposure. (B) T4302 CD 133+ cells were treated with GSIs ±radiation as indicated. Cells were then plated at 3000 cells per well in 96-well plates. Relative caspase 3/7 activities were determined by normalizing caspase activities to the corresponding cell titers. (C) T4302 CD 133+ cells were treated as described in FIG. 2A. 24 or 48 hours after radiation exposure, cells were labeled with FITC-conjugated Annexin V staining kit (BD Sciences) according to manufacturer's instructions and analyzed by flow cytometry. (D) T4302 CD 133+ cells were pre-treated with 2 μM DAPT for 4 hours, and were left unirradiated or irradiated at 1, 2 or 3 Gy. Relative caspase 3/7 activities were determined at 72 hours after radiation. (E) T4302 CD133+ cells were pre-treated with DAPT at indicated concentrations for 4 hours and irradiated at 3 Gy. Relative caspase activities were determined at 48 or 72 hours after radiation. *: p<0.01 by Student's t-test; #: p<0.01 by one-way ANOVA.

FIG. 3 is a series of graphs illustrating that the expression of the intracellular domains of Notch1 or Notch2 attenuates the radiosensitizing effects of GSIs. T4302 CD 133+ glioma cells were infected with control lentivirus or lentivirus directing expression of NICD1 or NICD2 and selected by puromycin. (A) Expression of Notch1, Notch2, Hes2, Hes4 and Hes5 were determined by quantitative real-time PCR. (B) Control cells or cells expressing NICD2 were transfected with Notch-dependent luciferase reporter and treated with DMSO or 2 μM DAPT over night. Notch-dependent transcription activities were determined by luciferase reporter assays as described in FIG. 1A. (C) Control cells or cells expressing NICDs were treated with DAPT ±radiation as indicated. Relative caspase 3/7 activities were determined at 48 or 72 hours after radiation, (D) apoptotic cell death was determined by Annexin V-staining as described in FIG. 2C, and (E) surviving fractions were determined as described in FIG. 1D. *: p<0.01 by Student's t-test; #: p<0.001, by one-way ANOVA.

FIG. 4 is an immunoblot and a graph that demonstrate that the Notch pathway does not alter DNA damage response of glioma stem cells. (A) T4302 CD133+ cells were pretreated with DPAT or L685,458 for 24 hours prior radiation. One hour after radiation exposure, cells were harvested. The levels of activating phosphorylation of Chk1 and Chk2 were assessed by immunoblotting. (B) T4302 CD133+ cells were treated with 2 μM DAPT at 4, 24 hours prior to radiation (−24 hrs, or -4 hrs), or at 0, 4, 24 hours after radiation. Relative caspase 3/7 activities were measured 3 days after radiation exposure. *: p<0.001 by Student's t-test.

FIG. 5 is a set of immunoblots and a graph illustrating that the Notch pathway regulates radioresistance of glioma stem cells via the Akt pathway and Mcl-1. T4302 CD 133+ cells were infected with control lentivirus or lentivirus directing expression of NICD2. Cells were pre-treated with or without 2 μM DPAT for 4 hours and exposed to 3-Gy radiation. Irradiated cells were collected at 24 or 48 hours after radiation, and sham-irradiated cells were collected at 48 hours after radiation. (A, C, D) Protein levels of phospho-Akt (S473), total Akt, PTEN, Bcl-2, Bcl-xL, Mcl-1, phospho-p53 (S15), total p53 and PUMA were assessed by immunoblotting. Actin was blotted as the loading control. (B) Control cells or NICD2-expressing cells were pre-treated with 10 μM PI3K inhibitor, LY294002, or 50 μM Akt inhibitor III (SH-6), for 4 hours prior to radiation exposure. Relative caspase 3/7 activities were determined at 48 hours after radiation exposure. #: p<0.001, by one-way ANOVA.

FIG. 6 is a series of graphs and an immunoblot illustrating that Notch inhibition by GSIs does not significantly alter radiation response of CD133-negative glioma cells. T3359 CD133-negative cells were pretreated with 2 μM GSI or 0.504 L685,458 for 4 hours and irradiated at 3 Gy or as indicated. (A) Relative caspase 3/7 activities were determined at 48 or 72 hour after radiation as described in FIG. 2B. (B) After radiation, cells in 6-well plates were cultured for three weeks, fixed and stained to count colonies. Surviving fractions were determined as described in methods. (C, D) The effects of GSIs on cell death and clonogenic survival of T4302 CD133-negative cells were determined as described above. ##: p>0.05 by one-way ANOVA. (E) T4302 CD133-negative cells were treated with GSIs ±radiation as described above. Levels of phospho-Akt (S473), total Akt, Mcl-1 and actin were determined by immunoblotting.

FIG. 7 is a series of graphs and an image illustrating that knockdown of Notch1 or Notch2 increases radiosensitivity of glioma stem cells in vitro and in vivo. T4302 CD 133+ cells were infected with lentivirus directing expression of non-targeting shRNA or shRNAs specific to Notch1 or Notch2 (KD-Notch1 or KD-Notch2), and selected with puromycin. (A) Relative mRNA levels of Notch1, Notch2, Hes4 or Hes5 were determined by quantitative real-time PCR. *: p<0.05 by Student's t-test. (B) Relative caspase 3/7 activities were determined in control cells and cells with Notch knockdown with or without radiation exposure as described in FIG. 2B. #: p<0.001 by one-way ANOVA. (C) Clonogenic survival was compared between the control group and cells with Notch knockdown as described in FIG. 1D. (D) Representative images (100×) of neurospheres formed by T4302 CD133+ cells with or without Notch knockdown two weeks after radiation. (E) After lentivirus infection and puromycin selection, T4302 CD133+ cells were left unirradiated or irradiated at 3 Gy, and were immediately injected into both flanks of athymic nude mice at 250,000 cells per injection. Three mice were inoculated for a total of six injections per group. Tumor growth was monitored twice a week for 4 weeks until diameters of the largest tumors reached approximately 20 mm.

FIG. 8 is a series of graphs illustrating that radiation activates Notch signaling in glioma stem cell. (A) T4302 CD133+ glioma stem cells were transfected with a control luciferase reporter (SABiosciences) and exposed to 3-Gy radiation at 2 days after transfection. Relative luciferase activities were determined as described in FIG. 1A. (B, C) Induction of Notch signaling was examined by luciferase reporter assays in glioma stem cells derived from the T3359 and T4105 short-term culture as described in FIG. 1A. (D-G) Following radiation, mRNA levels of Notch target genes, including Hes1, Hes2, Hes4 and Hes5, were determined in T3359 and T4302 glioma stem cells as described in FIG. 1B. *: p<0.05 by Student's t-test and the increase was more than 2 fold than the sham-irradiated groups.

FIG. 9 is a series of graphs illustrating that γ-secretase inhibitors impaired clonogenic survival of glioma stem cells following radiation. (A) T3359 glioma stem cells were GSIs ±radiation and then were plated at 200 cells per well in 24-well plates. The surviving fractions were determined as described in FIG. 1D. (B) T4597 glioma stem cells treated with GSIs and/or 3-Gy radiation were plated at 50 cells per well for the sham-irradiated groups and 500 cells per well for the irradiated groups. Neurospheres were scored in 14 days after radiation. #: p<0.001 by one-way ANOVA.

FIG. 10 is a series of graphs illustrating that γ-secretase inhibitors enhance radiation-induced cell death in glioma stem cells. (A, B) Relative caspase 3/7 activities of T3359 and T3691 glioma stem cells treated with GSIs and/or radiation were determined at 72 hours after radiation exposure as described in FIG. 2B. #: p<0.001 by one-way ANOVA.

FIG. 11 is a graph illustrating that expression of NICD2 protects T4105 glioma cells from radiation-induced cell death. T4105 glioma stem cells were infected with control lentivirus or lentivirus directing expression of NICD2. Cell death following 2 μM DAPT treatment and/or 3-Gy radiation exposure was determined by caspase 3/7 assays as described in FIG. 2B. *: p<0.001 by Student's t-test.

FIG. 12 is a graph illustrating that the Notch pathway does not alter DNA damage response of glioma stem cells. T4105 CD133+ cells were treated with 2 μM DAPT at different time points as described in FIG. 4B. Relative caspase 3/7 activities were measured 2 days after radiation exposure. *: p<0.001 by Student's t-test.

FIG. 13 is an immunoblot illustrating that LY294002 or Akt inhibitor III reduce Akt activity in glioma stem cells. T4302 glioma stem cells were treated with 10 μM LY294002 or 50 μM Akt inhibitor III (SH-6) in the presence or absence of radiation. Cells were collected for immunoblotting at 48 hours after radiation as described in FIG. 5.

FIG. 14 is a series of graphs illustrating that the knockdown of Notch1 or Notch2 increases radiosensitivity in glioma stem cells but not in CD133-glioma cells. T3359 glioma stem cells were infected with lentivirus directing expression of non-targeting shRNA or shRNAs specific to Notch1 or Notch2 (KD-Notch1 or KD-Notch2, 2b), and selected with puromycin. (A) relative caspase 3/7 activities were determined at 48 hours after radiation exposure as described in FIG. 2B. (B) Clonogenic survival was determined as described in FIG. 1E. (C) T4302 CD133-negative glioma cells were also infected with lentiviruses, and cell death was determined at 48 and 72 hours after radiation using caspase 3/7 assays as described in FIG. 2B. #: p<0.001 by one-way ANOVA.

### DETAILED DESCRIPTION OF PREFERRED EMBODIMENTS

The present invention is more particularly described below and particularly in the Examples set forth herein that are intended as illustrative only since numerous modifications and variations therein will be apparent to those skilled in the art.

As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practioner regarding the description of the invention.

The discovery that administration of GSIs can inhibit Notch signaling activity provides a mechanistic basis for treating gliomas and glioblastoma. However, the practice of the methods of this invention is not limited to any particular explanatory mechanism, but instead depends on the effects of administering GSIs, Notch signaling inhibitors or combinations thereof for treating gliomas and glioblastoma. In a particular embodiment, administration of GSIs or Notch signaling inhibitors increases sensitivity of gliomas to radiation therapy. In a preferred embodiment, a therapeutic effective amount of GSI is administered in association with a therapeutic effective amount of radiation.

Thus, in one aspect, the invention provides a method of increasing glioma sensitivity to radiation, wherein the method comprises administering a therapeutic effective amount of a GSI, Notch signaling inhibitor, or a combination thereof to a subject in need thereof. In a preferred embodiment, the gliomas and glioblastoma are radiation resistant. In an alternative embodiment, gliomas include glioma stem cells. In certain embodiments, the subject is a human, cat, dog, or mouse.

In another aspect, the invention relates to using GSIs, Notch signaling inhibitor, or a combination thereof for treating glioma and glioblastoma. In a preferred embodiment, the gliomas and glioblastoma are radiation resistant. In an alternative embodiment, the gliomas include glioma stem cells. In a further embodiment, treatment with a GSI, Notch signaling inhibitor, or a combination thereof inhibits Notch signaling activity, thereby rendering the glioma more susceptible to radiation therapy.

In preferred embodiments, the GSI (i.e., gamma secretase inhibitor) is DAPT (N-[N-(3,5-Difluorophenacetyl-L-alanyl)]-S-phenylglycine t-Butyl Ester)) (EMBD Biosciences, formerly Calbiochem), L685,458 (EMBD Biosciences), or GSI-18. The last proteolytic step that generates the active intracellular domains of Notch receptors is dependent on the γ-secretase complex [18, 19]. A substantial number of γ-secretase inhibitors have been developed for treatment of Alzheimer's disease, because the γ-secretase complex mediates production of β-amyloid peptides, the precursor of amyloid plaques found in Alzheimer's disease [75]. Given their activities of inhibiting Notch, GSIs have also been actively examined for their anti-cancer efficacy in a variety of tumor types, such as T-ALL [76], breast cancer [77], Kaposi's sarcoma 178], medulloblastorna [79], intestinal adenoma [23], etc. Clinical trials with MK0752, a γ-secretase inhibitor made by Merck, are underway in patients with breast cancer and pediatric central nervous system malignancies (clinicaltrials.gov). Studies provided herein demonstrate that GSIs alone only display limited growth inhibitory effects in gliomas, while in combination with radiotherapy, GSIs provide significant therapeutic benefits by promoting the radiosensitivity of glioma stem cells. It will be understood in the art that these three examples of GSIs are non-limiting, and that any compound having specific GSI activity fall within the scope of the methods and pharmaceutical compositions of the invention.

The phrase “inhibits Notch signaling” as used herein refers to the reduction Notch pathway specific signaling. The components and mechanisms or the Notch signaling pathway have been described herein. The blockade of Notch signaling in turn results in decreased Notch gene expression. A reduction in Notch cascade signaling by any means, including reduced gene expression, reduced protein translation, blockade of Notch nuclear translocation of the intracellular domains of Notch receptors (NICDs) are included within this definition.

As used herein the term “Notch signaling inhibitor” refers to a molecule, nucleotide, protein, antibody, etc. that is capable of inhibiting Notch signaling. Example of Notch signaling inhibitors include but are not limited to antisense RNA, shRNA, siRNA, antibodies, or dominant-negative forms of Notch 1 or Notch 2. The Notch pathway can promote or repress tumorigenesis in a context-dependent manner. For example, loss of Notch1 or expression of a dominant negative MAML1 mutant in mouse epidermis induces hyperplasia and subsequent skin tumors [64, 65]. In medulloblastoma, expression of NICD2 promotes tumor growth while NICD1 is tumor suppressive [60]. Conversely, knockdown Notch1 impaired proliferation and survival of glioma cell lines [61]. Using lentivirus-mediated expression of NICD1/2 or multiple Notch1/2-specific shRNAs, these suggest that both Notch1 and Notch2 promote radioresistance in glioma stem cells, although the relative contribution of each Notch receptor is undefined. These effects represent sustained inhibition of Notch by stable expression of shRNAs, off-target activities of the shRNAs, or oversaturation of the microRNA/short hairpin RNA pathways [66].

Notch1 is a key regulator of intestinal stem cell fate determination and gut homeostasis [22, 23]. Clinical trials using GSIs for treatment of Alzheimer's disease are primarily associated with gastrointestinal toxicity [68, 69], which is likely due to inhibition of Notch1 by GSIs. If Notch receptors other than Notch1 are also essentially required for radioresistance of glioma stem cells, small molecules or antibodies that specifically target these receptors while sparing Notch1 will achieve similar radiosensitizing effects in gliomas with reduced gut toxicity. It will be understood in the art that these three examples of are non-limiting, and that any molecule, nucleotide, protein, or antibody having specific Notch signaling inhibitory effect fall within the scope of the methods and pharmaceutical compositions of the invention.

As used herein, the term “radiation resistant” refers to cells and in particular cancer cells that are not readily killed by radiation therapy. Examples of such cells are provided herein, including radiation resistant glioma stem cells. The phrase “increasing the sensitivity of gliomas to radiation” as used herein refers to modulating glioma cells to have increased susceptibility to radiation therapy (e.g., increased rates of cell death). In a particular embodiment, this is accomplished by the administration of a gamma secretase inhibitor or a Notch signaling inhibitor.

As used herein, the terms “glioma” or “malignant glioma” include but are not limited malignant gliomas (of any grade), and further include but are not limited to anaplastic astrocytoma or anaplastic glioma (World Health Organization (WHO) grade III) and glioblastoma multiforme (WHO grade IV). The term “glioma stem cell” as used herein refers to glioma cells that express stem cell representative cell surface markers. Cancer cells with stem cell-like properties have been described in a wide range of human tumors. The cancer stem cells model suggests a hierarchical organization of tumors that a subpopulation of tumor cells at the apex drives and maintains human tumors [5]. Cancer stem cells of glioma and other neoplasms of human central nervous system can be prospectively enriched by selection of the CD133 (prominin-1) cell surface marker [6,7,8,9,10,11], although some tumors may not express CD133 and hence CD133-negative cells may still have characteristics of cancer stem cells [12,13]. Despite expression of certain neural and other stem cell markers (such as CD133, Musashi-1, Nestin, Sox2 and Olig2) by brain tumor stem cells, the definition of cancer stem cells remains functional, requiring sustained self renewal and tumor propagation.

The terms “effective amount” or “therapeutically effective amount” refer to the amount of a compound including a GSI, Notch signaling inhibitor, or a combination thereof that is effective, upon single or multiple dose administration to a patient, in treating the patient suffering from the glioma or glioblastoma. The term “treatment” or “treating” is intended to encompass also prophylaxis, therapy and cure.

A “patient” or “subject” to be treated by the subject method can mean either a human or non-human animal, including but not limited to a dog, cat, or mouse.

In another aspect, the invention provides pharmaceutical preparations comprising GSIs, Notch signaling inhibitors or combinations thereof adapted for use in the inventive methods disclosed herein. GSIs for use in the methods of the invention can be conveniently formulated for administration with a biologically acceptable, non-pyrogenic, and/or sterile medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the GSI, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

Pharmaceutical formulations of the present invention can also include veterinary compositions, e.g., pharmaceutical preparations of the GSI suitable for veterinary uses, e.g., for the treatment of mammals and domestic animals.

The pharmaceutical compositions, formulations and preparations of the invention can be given orally, parenterally, topically, or rectally. They are, of course, given by forms suitable for the desired administration route. For example, they can be administered in tablets or capsule form, by injection, inhalation, ointment, suppository, controlled release patch, administration by injection, infusion or inhalation; topical by lotion or ointment; and by rectal suppositories. Oral and topical administrations are preferred.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. The selected dosage level will depend upon a variety of factors including the activity of the particular compound employed, or the ester, salt or amide thereof, the route of administration, the time of administration, the rate of excretion of the particular compound being employed, the duration of the treatment, other drugs, compounds and/or materials used in combination with the particular reuptake inhibitors employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

If desired, the effective daily dose of the active compound may be administered as two, three, four, five, six or more sub-doses administered separately at appropriate intervals throughout the day, optionally, in unit dosage forms.

While it is possible for a compound of the invention to be administered alone, it is preferable to administer the compound as a pharmaceutical formulation (composition). Pharmaceutical compositions according to the invention may be formulated for administration in any convenient way for use in human or veterinary medicine.

Thus, another aspect of the invention provides pharmaceutically-acceptable compositions or formulations comprising a therapeutically effective amount of one or more of the GSI compounds described herein, formulated together with one or more pharmaceutically acceptable carriers (additives) and/or diluents. As described in detail herein, pharmaceutical compositions of the invention can be specially formulated for administration in solid, liquid, or sustained release form, including those adapted for the following: (1) oral administration, for example, drenches (aqueous or non-aqueous solutions or suspensions), tablets, boluses, powders, granules, and pastes for application to the tongue; (2) parenteral administration, for example, by subcutaneous, intramuscular, or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile liquid.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary, paste, or a sustained released or delayed release formulation.

A composition of the invention comprising a polypeptide, polynucleotide, antibody, or a combination thereof is administered in a manner compatible with the particular composition used and in an amount that is effective to elicit an immune response as detected by, for example, an ELISA. A polynucleotide can be injected intramuscularly to a mammal, such as a dog, or human, at a dose of 1 ng/kg, 10 ng/kg, 100 ng/kg, 1000 ng/kg, 0.001 mg/kg, 0.1 mg/kg, or 0.5 mg/kg. A polypeptide or antibody can be injected intramuscularly to a mammal at a dose of 0.01, 0.05, 0.5, 0.75, 1.0, 1.5, 2.0, 2.5, 5 or 10 mg/kg.

Polypeptides, polynucleotides, or antibodies, or a combination thereof can be administered either to an animal that is not infected with E. chaffeensis or can be administered to an E. chaffeensis-infected animal. An immunologically effective amount or therapeutically effective amount means the administration of that amount to an individual, either in a single dose or as part of series, is effective for treatment, amelioration, or prevention of E. chaffeensis infection. The particular dosages of polynucleotide, polypeptides, or antibodies in a composition will depend on many factors including, but not limited to the species, age, gender, concurrent medication, general condition of the mammal to which the composition is administered, and the mode of administration of the composition. An effective amount of the composition of the invention can be readily determined using only routine experimentation.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

The Examples which follow are illustrative of specific embodiments of the invention, and various uses thereof. They set forth for explanatory purposes only, and are not to be taken as limiting the invention.

### Example 1

### γ-Secretase Inhibitors Impair Cell Growth and Clonogenic Survival of Glioma Stem Cells in Combination with Radiation

Accumulating evidence suggests that the Notch pathway plays important roles in a wide range of human tumors as well as cancer stem cells [31, 41]. It has been reported that radiation induces transient upregulation of Notch pathway components in endothelial cells [42]. To determine the role of Notch activity in radiation response of Omit stem cells, the following studies were performed.

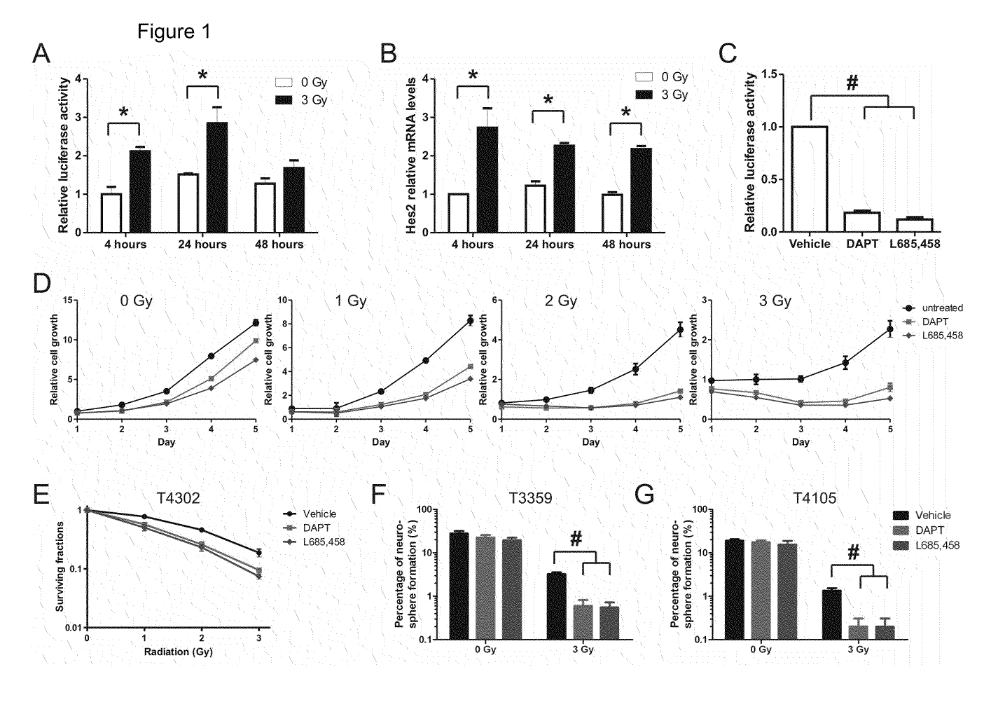

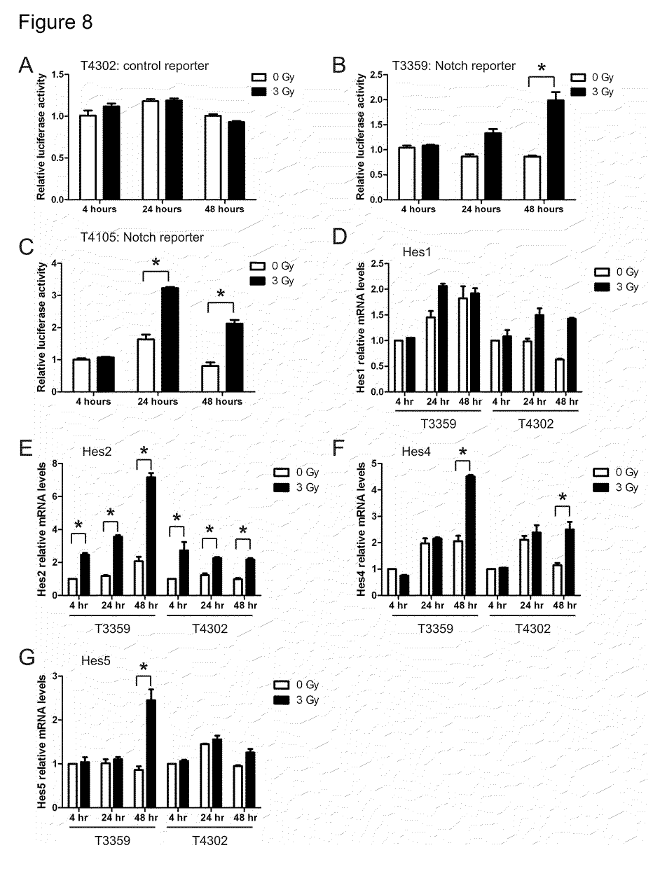

Short-term cultures enriched for glioma stem cells were isolated from surgical biopsy specimens and amplified as direct xenografts that have been demonstrated to maintain a cancer stem cell phenotype [39]. The stem cell-like properties of the CD133+ glioma cells specifically used in this study, including sustained self-renewal, expression of stem cell markers, multi-lineage differentiation, and the ability to recapitulate the original tumors in serial xenotransplantation assays, have been demonstrated in our previous publications [6, 7, 36-38, 40]. In T3359, T4105, and T4302 CD133+ glioma cells, radiation induced modest transcription activation from a Notch-responsive luciferase reporter (RBP-Jκ reporter, SABiosciences) but not from a control reporter (SABiosciences) (FIG. 1A and FIG. 8A-C). Expression of Notch target genes, including Hes2, and to a lesser degree, Hes4 and Hes5, was increased after radiation exposure (FIG. 1B and FIG. 8D-U). Notch signaling in glioma stem cells was blocked by γ-secretase inhibitors, DAPT (EMD Biosciences) or L685,458 (EMD Biosciences) as shown by Notch-responsive luciferase reporter assays (FIG. 1C). Using CD133+ glioma cells derived from T4302 and other glioma sources, including T3359, T4105 and T4597 (FIG. 1D), these experiments showed that GSIs moderately reduced growth of CD 133+ glioma cells in the absence of radiation (FIG. 1D, graph 1) but severely impaired growth of cells exposed to radiation at either 1, 2 or 3 Gy (FIG. 1D, graph 2-4). On the fifth day after 2-Gy radiation, total cell numbers in the sham-treated groups were approximately 4 fold more than in GSI-treated groups. In contrast, without radiation treatment, sham-treated cells were only 25% (vs. DAPT-treated) or 60% (vs. L685,458-treated) more than GSI-treated cells five days after radiation.

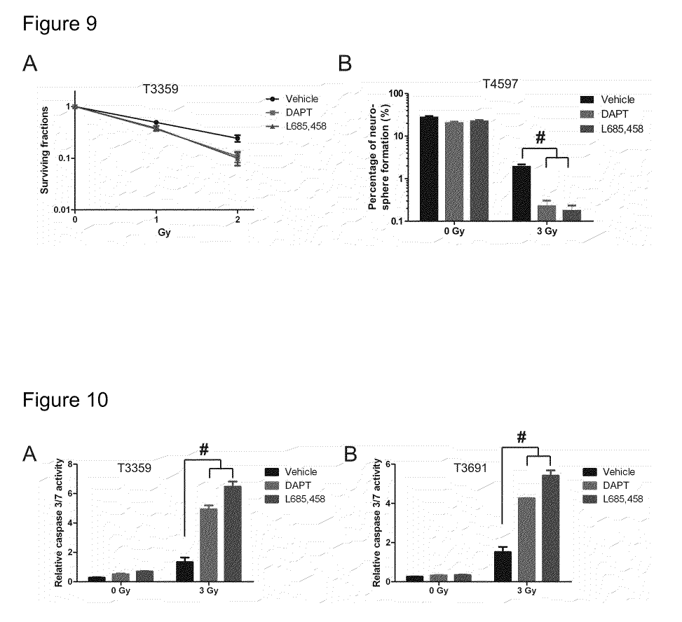

Clonogenic survival assay is the standard assay to assess radiation responsiveness in vitro. In this study, neurosphere formation as a surrogate marker of the clonogenic survival of glioma stem cells was utilized. In combination with radiation, GSIs significantly reduced clonogenic survival of the CD133+ glioma cells compared with the sham-treated groups for all radiation doses examined (FIG. 1E, p=0.012 at 1 Gy, p<0.001 at 2 and 3 Gy, by one-way ANOVA). Similar results were demonstrated using CD133+ cells derived from other glioma sources, such as T3359, T4105 and T4597 (FIGS. 1F, 1G, and FIGS. 9A, 9B). Taken together, these results demonstrate that Notch inhibition with γ-secretase inhibitors compromises radioresistance of glioma stem cells.

### Example 2

### γ-Secretase Inhibitors Enhance Radiation-Induced Cell Death in Glioma Stem Cells in Combination with Radiation

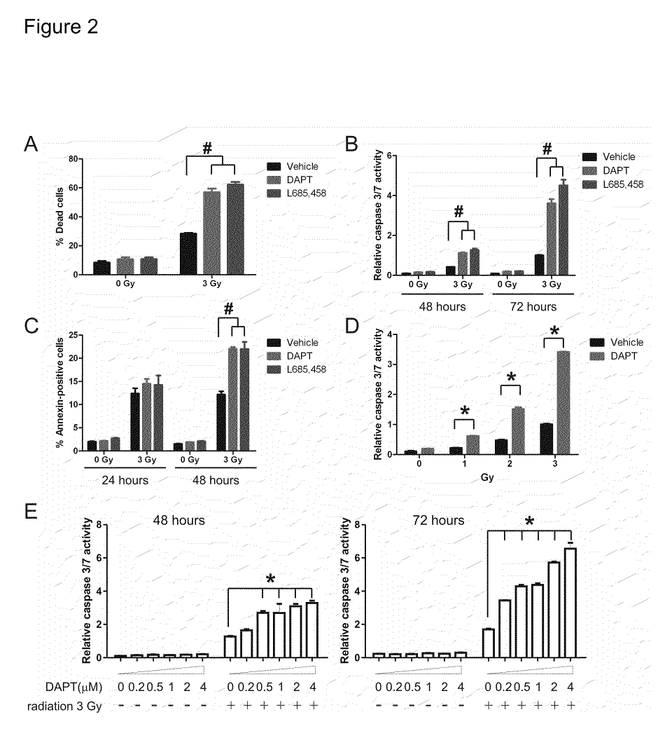

Significantly greater radiation-induced cell death was observed in GSI-treated cells compared with sham-treated cells. Two days after 3-Gy radiation, sham-treated T4302 CDI33+ cells contained approximately 30% dead cells, whereas 60% of cells were dead in the presence of DAPT or L685,458 (FIG. 2A). Conversely, GSIs did not have an obvious impact on the percentage of dead cells in the absence of radiation (FIG. 2A). The majority of GSI-treated cells died within 72 hours after 3 Gy of irradiation.

Activation of caspase is a hallmark of apoptotic cell death and is critically involved in radioresponse [43-46]. Using luminescence-based caspase-3/7 assays, we examined how Notch inhibition affected radiation-induced caspase activation as a surrogate marker of cell death. GSIs alone did not significantly alter caspase activities in the sham-irradiated T4302 CD 133+ cells (FIG. 2B, p>0.05, by one-way ANOVA). In contrast, combining GSIs with 3-Gy radiation increased caspase activities by approximately 2 fold at 48 hours and 4 fold at 72 hours (FIG. 2B, p<0.01 by one-way ANOVA). Similar results were detected using CD133+ glioma cells derived from several glioma sources, including T3359, T3691, T4105 and T4597 (FIG. 10). In validation studies, apoptotic cell death was assessed by flow cytometry analysis of Annexin V, a cell surface marker for apoptosis. Both DAPT and L685,458 significantly increased Annexin V-positive labeled cells at 48 hours following radiation (FIG. 2C, p<0.01 by one-way ANOVA), although the effects were not obvious at 24 hours following radiation (FIG. 2C, p>0.05 by one-way ANOVA). Additionally, DAFT enhanced radiation-induced cell death at different radiation doses (FIG. 2D). The radiosensitizing effects of DAPT showed a concentration-dependent effect, with 200 nM DAPT (IC50 as suggested by the manufacturer) still being effective (FIG. 2E). Collectively, these data demonstrate that Notch inhibition using γ-secretase inhibitors sensitize glioma stem cells to radiation-induced cell death.

### Example 3

### Expression of the Constitutively Active Intracellular Domains of Notch1 or Notch2 Attenuates the Radiosensitizing Effects of GSIs

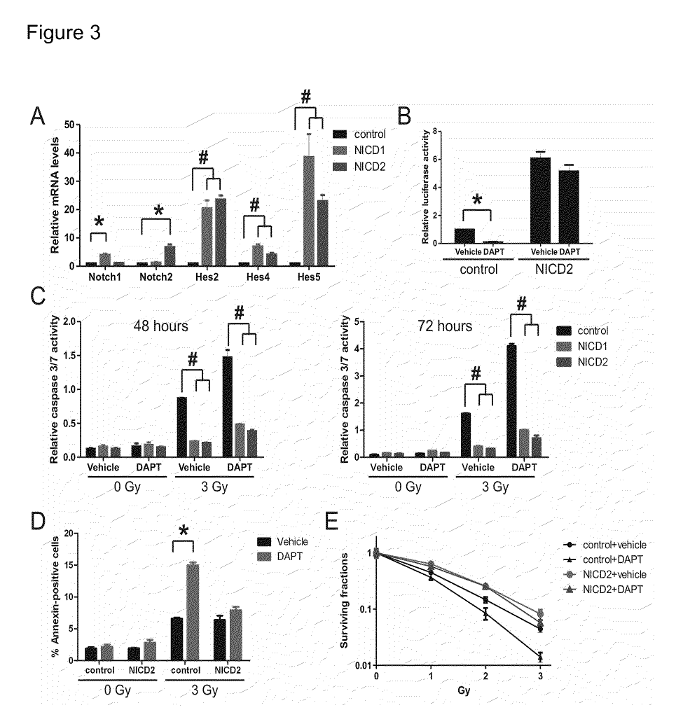

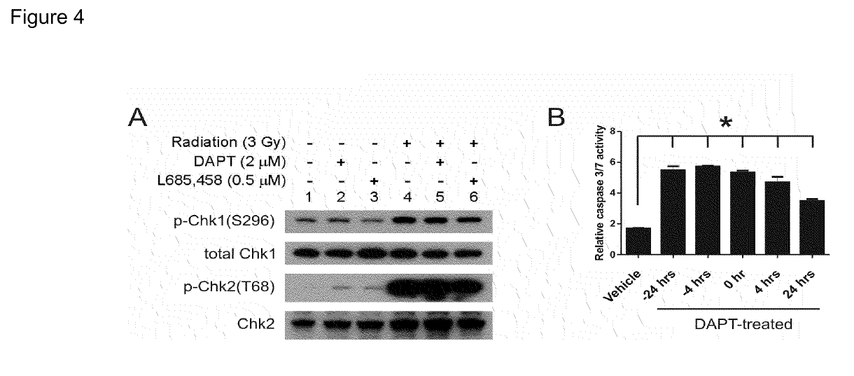

Substrates of GSIs include more than fifty type I membrane proteins in addition to the Notch receptors [47]. The radiosensitizing effects of GSIs might not be necessarily mediated through inhibition of Notch. Studies were performed to elucidate the role of Notch in radioresponse of glioma stem cells via the examination of expression of the constitutively active intracellular domains of Notch1 or Notch2 (NICD1 or NICD2) that function downstream of GSIs. Activation of the Notch pathway by lentivirus-mediated expression of NICD1 or NICD2 was validated by upregulation of Notch target genes, including Hes2, Hes4 and Hes5 (FIG. 3A), as well as increased luciferase activities generated from the Notch-responsive reporter (FIG. 3B). Similar to the parental cells, cells infected with the control lentivirus were sensitive to DAPT when irradiated (FIG. 3C). In contrast, cells expressing either NICDI or NICD2 demonstrated significantly weaker radiation-induced cell death in comparison to control cells regardless of DAPT treatment (FIG. 3C). Additionally, DAPT treatment failed to increase the percentage of Annexin V-positive cells in the presence of NICD2 expression (FIG. 3D). Finally, expression of NICD2 improved clonogenic survival of the T4302 glioma stem cells following radiation regardless of DAPT treatment (FIG. 4E, p=0.004, NICD2 vs. control ±DAPT; p<0.01, NICD2 DAPT vs. control ±DAPT, at 3-Gy radiation by one-way ANOVA), whereas DAPT treatment did not significantly alter clonogenic survival of irradiated NICD2-expressing cells (FIG. 4E, p>0.05, NICD2 vs. NICD2-FDAPT, by Student's t-test).

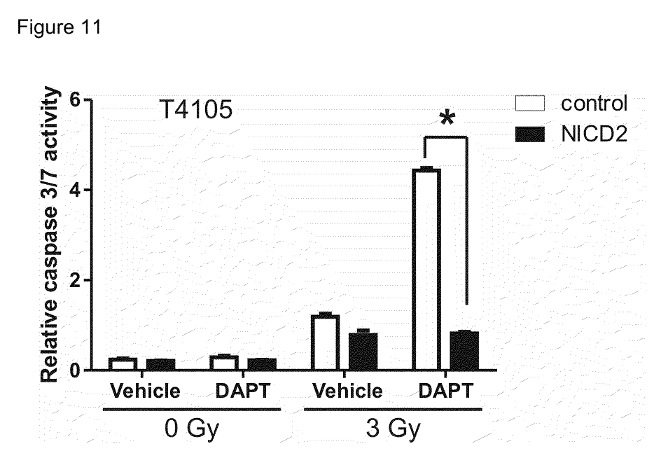

Additionally, expression of N1CD1 or NICD2 protected glioma stem cells derived from T3359 and T4105 in a similar manner (FIG. 11). Taken together, these data demonstrate that Notch activation by expression of NICDs attenuates the radiosensitizing effects of GSIs, illustrating that these activities of GSIs are crucially mediated through inhibition of Notch.

### Example 4

### Notch Pathway Fails to Alter DNA Damage Response of Glioma Stem Cells, but Notch Promotes Radioresistance Through Regulation of the PI3K/Akt Pathway and Mcl-I

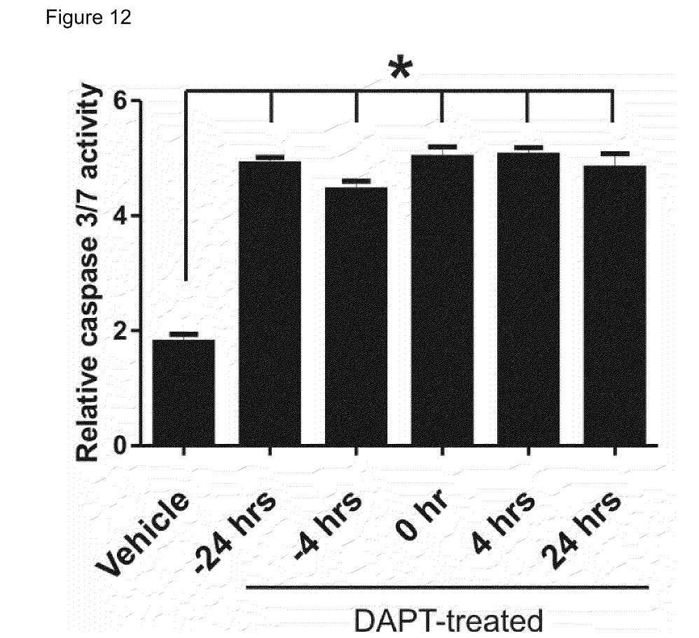

The DNA damage checkpoint response plays a critical role in cellular response toward radiation [48, 49]. Previous studies by this laboratory showed that increased activation of the DNA damage response is implicated in radioresistance of the glioma stem cells [7]. To elucidate the mechanisms through which Notch promotes radioresistance of glioma stem cells, it was determined whether the Notch pathway affected activation of the checkpoint kinases following radiation. Neither of DAPT or L685,458 significantly altered activating phosphorylation of Chk 1 or Chk2 following radiation (FIG. 4A). Additionally, in either T4105 or T4302 CD133+ glioma cells, DAPT increased radiation-induced cell death even when it was added 24 hours after radiation (FIG. 4B and FIG. 12), whereas the bulk DNA damage repair process is usually completed within a few hours after radiation exposure [50]. These results show that the DNA damage response pathway is not critically involved in the radioprotective activities of Notch.

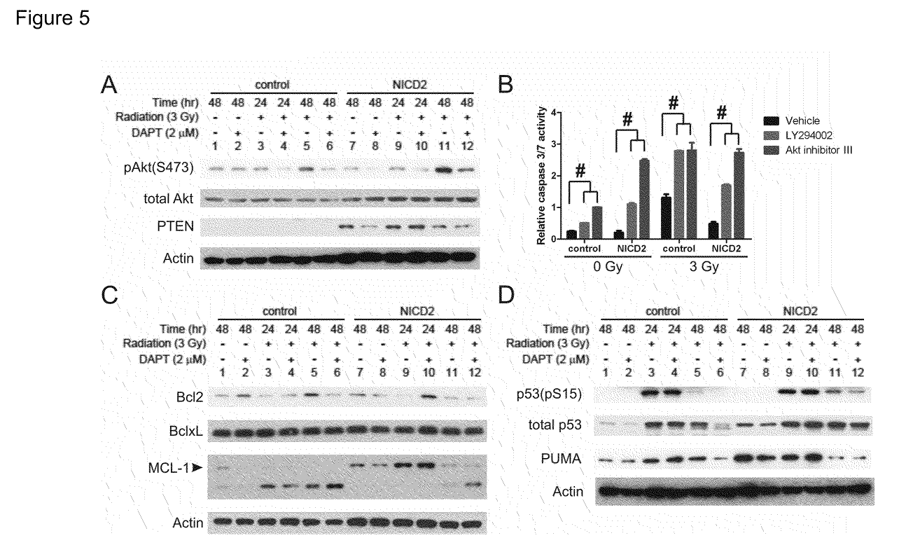

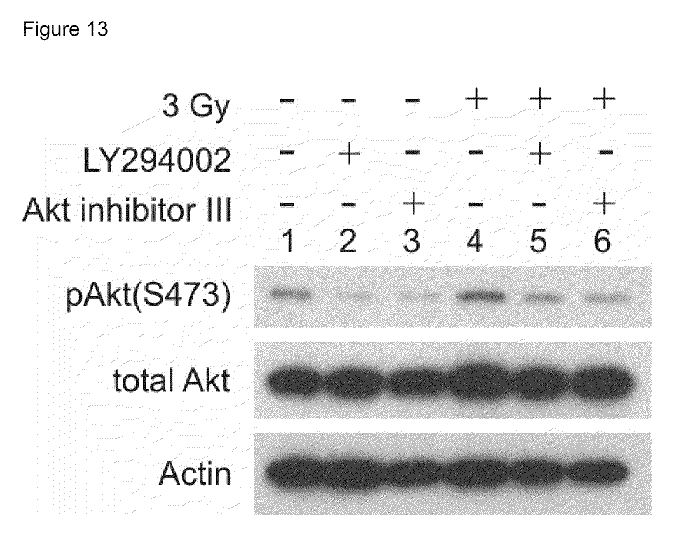

Notch inhibition renders glioma stem cells more sensitive to radiation-induced cell death. In a context-dependent manner, Notch can promote cellular survival through different mechanisms, such as activation of the PI3K/Akt pathway [51, 52] and upregulation of the prosurvival proteins, Bcl-2 and Mcl-1 [53]. To determine the pathway by which Notch mediates radioresistance, alterations of these prosurvival factors between the NICD2-expressing glioma stem cells and the control cells treated with DPAT, radiation, or both were assessed. Activating phosphorylation at serine 473 of Akt was used as a surrogate marker of Akt activity. While radiation exposure induced Akt activation as shown by increased levels of phospho-S473 Akt, DAPT treatment reduced Akt activation (FIG. 5A). In contrast, expression of NICD2 increased Akt activities, which was partially attenuated but not abolished by DAPT (FIG. 5A). Similar results were found in other short term glioma cultures, such as 13359 and T4105. The inventors recently reported that Akt activity was essential for survival of glioma stem cells [37]. In accordance with this finding, treatment of a PI3K inhibitor, LY294002, or the Akt inhibitor III, increased cell death of glioma stem cells with or without radiation (FIG. 5B). Treatment of LY294002 or the Akt inhibitor reduced phosphorylation at S473 of Akt (FIG. 13). Expression of NICD2 did not overcome the apoptotic effects of either LY294002 or the Akt inhibitor III, suggesting that the PI3K/Akt pathway functions downstream of Notch (FIG. 5B).

The anti-apoptotic Bcl-2 proteins, such as Bcl-2, Bcl-xL and Mcl-1, are important promoters for cell survival [54]. The levels of Bcl-2 and Bcl-xL were not apparently altered by either radiation or Notch alteration (FIG. 5C). Conversely, radiation exposure or DAPT treatment decreased the levels of Mcl-1, and expression of NICD2 increased Mcl-1 levels even in the presence of DAPT (FIG. 5C). Interestingly, radiation or DAPT increased the levels of Mcl-1 proteins of a smaller size (FIG. 5C). These bands could represent either proteolytically cleaved Mcl-1 proteins generated during apoptosis or a truncated isoform of Mcl-1 (Mcl-10, which is pro-apoptotic due to loss of BH I and BH2 domains [55].

The functional interaction between Notch and p53 is intriguing, as Notch can activate p53 [56, 57] or inhibit p53 [58] in a cell type-specific manner. As a crucial regulator of cellular radioresponse, p53 activity may be involved in the radioprotective function of Notch. These studies showed that expression of NICD2 increased p53 levels in the absence of radiation, and further enhanced p53 activation induced by radiation (FIG. 5D). Additionally, expression of p53 target gene, PUMA, was upregulated by NICD2 (FIG. 5D). These results are consistent with previous study showing that Notch upregulates p53 in gliomas [57], suggesting that the radioprotective functions of Notch are not mediated by inhibition of p53.

These results demonstrate that Notch inhibition in irradiated glioma stem cells dramatically increased cell death occurring within 3 days after radiation, suggesting that Notch is crucially involved in post-radiation survival of glioma stem cells. In accordance with these observations, Notch upregulates the prosurvival factors Akt and Mcl-I in glioma stem cells. It has been recently reported that Mcl-1 is a critical regulator of survival in neural precursor cells during development and in response to DNA damage [53, 70], Akt is known to upregulate Mcl-1 levels [71, 72]. Therefore, the regulation of Mcl-1 by Notch can be mediated through Akt. The P13K/Akt pathway plays a central role in the radioprotective functions of Notch, as shown by the results that inhibitors of PI3K or Akt abolish the radioprotective activities of NICD2. This pathway has profound functions in many aspects of cell growth, proliferation and survival [73]. We previously described an important link between the PI3K/Akt pathway with growth and survival of glioma stem cells [37]. Confirmation of these findings came from studies of a mouse medulloblastoma model in which the PI3K/Akt pathway is activated in medulloblastoma stem cells following radiation and is essentially required for post-radiation cell survival [74]. Additionally, inhibition of Akt sensitizes the medulloblastoma stem cells to radiation [74]. The mechanisms through which Notch activates the PI3K/Akt pathway in glioma stem cells remain to be elucidated. Although Notch can activate Akt through negatively regulate PTEN in T-cell acute lymphoblastic leukemia (T-ALL), experimental results herein showed that expression of NICD2 upregulated PTEN irrespective of radiation exposure, illustrating that Notch does not activate the PI3K/Akt pathway by repressing PTEN.

Alternatively, a Src-related tyrosine kinase, p561ck, has been reported to mediate Notch-induced Akt activation [52]. Data disclosed herein show that neither a p561ck-specific inhibitor nor a pan-Src family kinase inhibitor altered radiation response of glioma stem cells indicating that Src tyrosine kinases are not implicated in radiation response of glioma stem cells. A recent study reports a non-canonical transcription-independent Notch function that membrane-anchored Notch1 is sufficient to activate Akt [67]. This novel mechanism may be implicated in Akt activation by Notch in glioma stem cells, but details of this functional interaction remain to be elucidated. Data provided herein shows that NICD2 upregulates PTEN and p53 in glioma stem cells, instead of repressing these two tumor suppressors, indicating that these two proteins are not be directly involved in the radioprotective functions of Notch. However, post-radiation survival of medulloblastoma stem cells can be promoted by cell cycle arrest mediated through PTEN and p53 [74]. Therefore, in glioma stem cells, Notch-induced PTEN and p53 can indirectly contribute to reduced cell death after radiation by promoting cell cycle arrest. Taken together, these studies illustrate a Notch-regulated signaling pathway that is critically involved in radiation response of glioma stem cells. This pathway provides multiple therapeutic targets at several levels, including the Notch receptors, the kinases (PI3K, Akt), and Mcl-1, a molecule that directly regulates the apoptosis cascade.

### Example 5

### CD 133-Negative Glioma Cells Do Not Respond to γ-Secretase Inhibitors

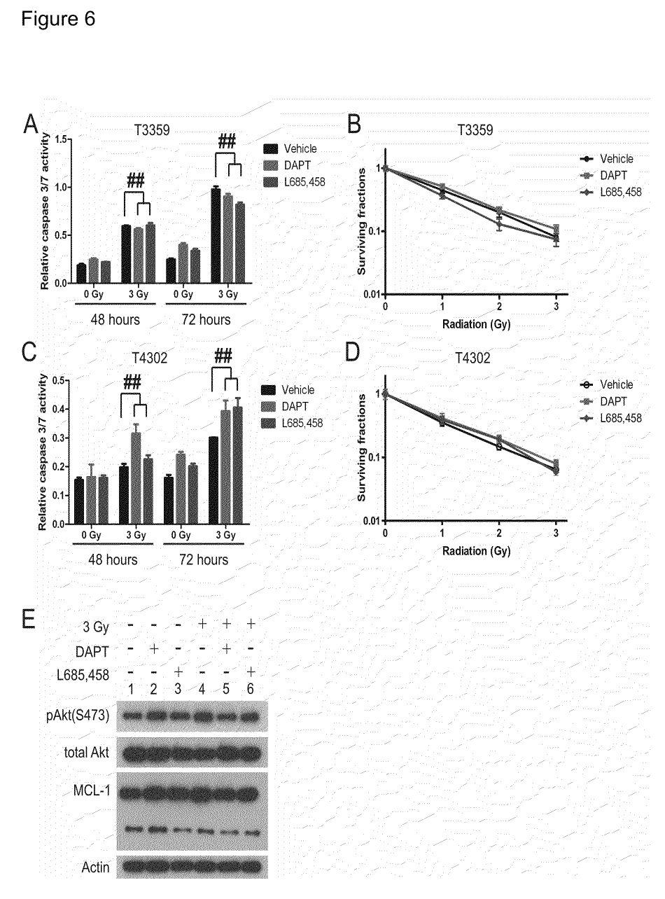

Studies were performed to determine whether Notch inhibition affected radioresponse of the CD 133-negative fraction of gliomas. Following similar procedures, cell death and clonogenic survival after radiation exposure were determined in the T3359 and T4302 CD 133-negative cells with or without GSI treatment. In contrast to glioma stem cells, caspase activation or clonogenic survival of the CD133-negative glioma cells were not significantly altered by Notch inhibition using GSIs irrespective of radiation exposure (FIG. 6A-D). Additionally, GSI treatment did not alter Akt activities or Mcl-1 levels in the CD 133-negative glioma cells (FIG. 6E). Taken together, our data show that the radiosensitizing effects of Notch inhibition are specific to the cancer stem cell fraction of gliomas.

The oncogenic role of Notch is highlighted by the presence of mutations of the Notch pathway components in a variety of human tumors, in particular leukemia and breast cancer [63]. Upregulation of Notch pathway components also presents in glioblastorna multiforme cell lines and surgical biopsy specimens [30]. A critical role of the Notch pathway in the maintenance of brain tumor stem cells has recently emerged [41]. Experimental studies provided herein demonstrate that the radioprotective functions of Notch are specific to the CD 133+ glioma stem cells. Neither cell death nor clonogenic survival of the non-stem glioma cells are significantly altered by GSI treatment irrespective of radiation exposure.

### Example 6

### Knockdown of Notch1 or Notch2 Sensitizes Glioma Stem Cells to Radiation and Impaired Xenograft Tumor Formation

Individual Notch receptors may have distinct biological functions in certain tumor types [59]. In medulloblastoma, it has been demonstrated that expression of the constitutively intracellular regions of Notch1 or Notch2 appears to exert opposite effects, with Notch1 acting as tumor suppressor and Notch2 acting as tumor promoter [60]. In gliomas, knockdown of Notch 1 impairs cell growth and survival [61]. By expression of NICD1 or NICD2, current studies suggest that both Notch receptors promote radioresistance of glioma stem cells. To gain a better understanding of how individual Notch receptors are involved in radioresponse, the effects of knockdown of Notch1 or Notch2 on radioresistance through lentivirus-mediated expression of shRNA sequences targeting Notch1 or Notch2 was determined.

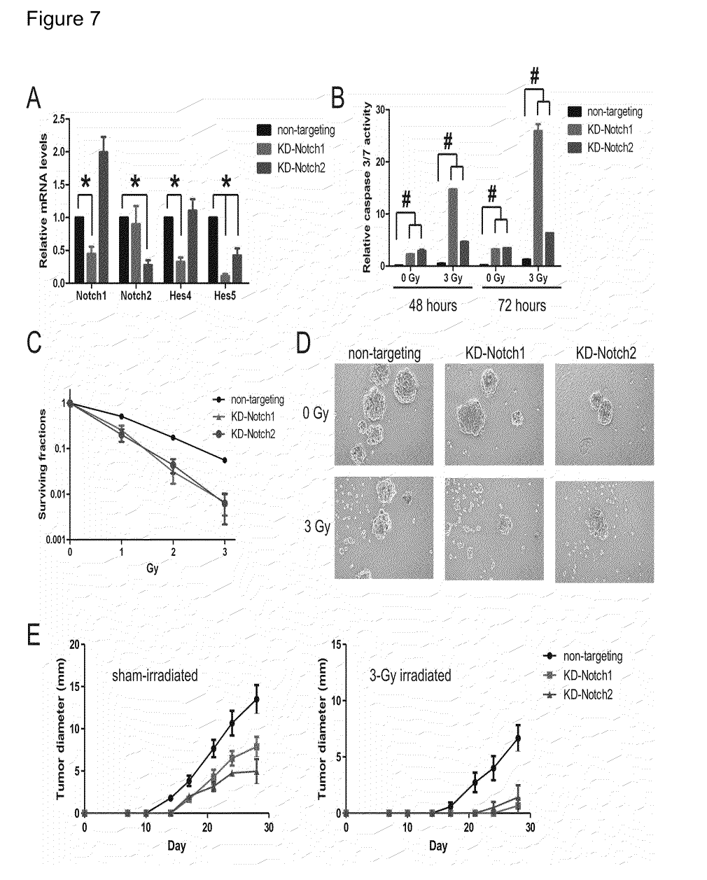

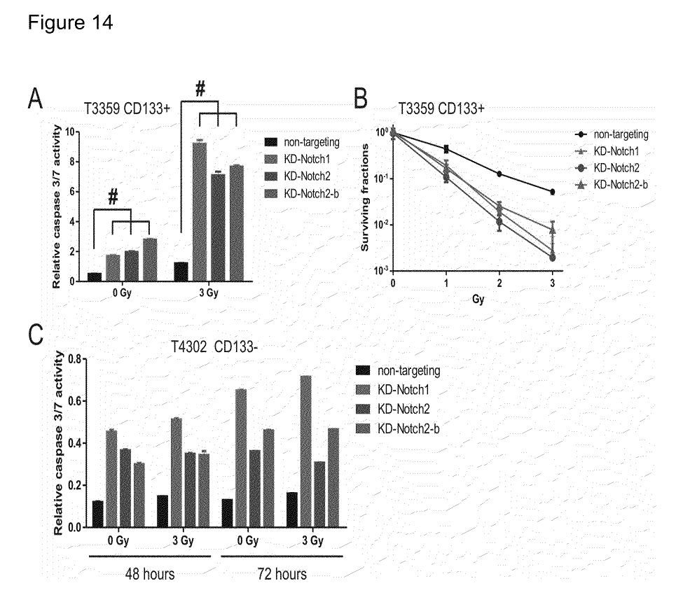

The efficiency of downregulation of the corresponding Notch receptors and Notch target genes were determined by quantitative real-time PCR (FIG. 7A). Knockdown of Notch1 alone increased apoptotic cell death in T3359 and T4302 CD133+ cells. Interestingly, similar effects were achieved by knockdown of Notch2 (FIG. 7B and FIG. 14). Cells with reduced Notch1 or Notch2 expression were highly sensitive to radiation-induced cell death (FIG. 7B and FIG. 14A). Consistently, the post-radiation clonogenic survival of glioma stem cells was severely impaired by knockdown of either Notch1 or Notch2 (FIG. 7C and FIG. 14B). When exposed to 3-Gy radiation, glioma stem cells expressing shRNA specific to Notch1 or Notch2 rarely formed neurospheres (FIGS. 7C and 7D). Additionally, knockdown of Notch1 or Notch2 alone reduced cell viability of the CD 133-negative glioma cells, but did not significantly increase radiation-induced cell death of these cells (FIG. 14C, p>0.05, irradiated vs. sham-irradiated by Student's t-test). These results further validate the specific role of Notch pathway in regulation of radioresistance, and indicate that multiple Notch receptors are likely involved in radiation response of glioma stem cells.

In vivo studies were performed to examine how impaired Notch signaling affected radiation response of glioma stem cells in vivo using a flank xenograft model. Control T4302 CD133+ glioma cells or cells with reduced Notch1 or Notch2 expression were subcutaneously inoculated into both flanks of nude mice without radiation exposure or immediately after 3-Gy radiation exposure. The sham-irradiated control cells formed palpable tumors at all injection sites at day 14 after injection (FIG. 7E and Table 1).

TABLE 1
Tumor incidence of T4302 glioma stem cells treated with a radiation ±
Notch shRNA. Xenograft assays were performed as described in FIG.
7E. In the irradiated group, p = 0.0094 by Chi-square test.

| Radiation | | | |
|---|---|---|---|
| dose | Non-targeting | KD-Notch1 | KD-Notch2 |
| 0 Gy | 6/6 | 6/6 | 5/6 |
| 3 GY | 6/6 | 1/6 | 2/6 |

Knockdown of either Notch1 or Notch2 alone modestly extended tumor latency (FIG. 7E). Tumors formed by irradiated control cells were first detected at day 17, and all became palpable at day 21 (FIG. 7E). In combination with radiation, knockdown of Notch1 or Notch2 not only extended the tumor latency (FIG. 7E), but also significantly decreased tumor incidence (1/6 for knockdown of Notch 1, and 2/6 for knockdown of Notch2, (Table 1). Taken together, these results demonstrate that integrated Notch signaling is critically implicated in radiation resistance of glioma stem cells in vivo. Knockdown of Notch1 or Notch2 modestly impaired cell viability in the non-stem glioma cells, but did not alter radiation-induced cell death. These studies demonstrate that stem cell-specific pathways, such as Notch, can serve as therapeutic targets.

### Example 7

### Materials and Methods

Enrichment of glioma stem cells and cell culture. Matched cultures enriched or depleted for glioma stem cells were isolated from human surgical specimens (T3359, T3691, T4105, T4302, and T4597) that were immediately implanted in immunocompromised mice (athymic BALB/c nude) according to a method that has been described in our previous studies and those of others to preserve cancer stem cells in glioma models [7,36,37,38,39,40]. Briefly, tumors were dissected, washed in Earle's balanced salt solution, digested with papain (Worthington Biochemical, Lakewood, N.J.) and filtered through 70 μm cell strainer to remove tissue pieces. Red blood cells were lysed in diluted phosphate buffered saline solution (0.25×). Dissociated cells were then cultured overnight in stem cell media (neurobasal media supplemented with B27, epidermal growth factor and basic fibroblast growth factor at 20 ng/ml, (Invitrogen, Carlsbad, Calif.) prior to cell sorting for recovery of cellular surface antigens. The CD133− and CD133+ fractions were separated by magnetic sorting using the CD133 Microbead kit (Miltenyi Biotec, Auburn, Calif.). CD133− cells were maintained in Dulbecco's modified Eagle's medium (DMEM) with 10% fetal bovine serum (Invitrogen), but were cultured in stem cell media at least 24 hours prior to experiments to control differences in cell media. The cancer stem cell properties of the CD133 positive cells was confirmed by fluorescent in situ hybridization, serial neurosphere assays, and xenotransplantation assays, but cultures depleted of cancer stem cells did not initiate tumors ([7,36,37,38,39,40] and data not shown).

Antibodies, DNA constructs, lentiviruses and other reagents. The antibodies purchased from Cell Signaling Technology (Danvers, M A) include phosphor-S296-Chk1 (2349), total Chk1 (2345), phosphor-T68-Chk2 (2661), total Chk2 (2662), phospho-5473 Akt (#9271), total Akt (#9272), PTEN (#9556), phospho-S15-p53 (#9286), Bcl-2 (#2870), Bcl-xL (#2764), Mcl-1 (#4572), Puma (#4976). Other antibodies used in this study are p53 antibody (#sc-126, Santa Cruz Biotechnology, Santa Cruz, Calif.), and actin antibody (Millipore, Billerica, Mass.). The pcDNA3-Notch1 construct was kindly provided by Dr. Spyros Artavanis-Tsakonas, Harvard University. The coding sequences of intracellular domain of Notch1 (amino acid residues 1744-2556) was amplified by PCR and subcloned into the lentiviral vector pCDH-CMV-EF1-puro (System Biosciences, Mountain View, Calif.) through the EcoRI and NotI sites. The pcDNA3-NICD2 is a kind gift of Dr. Charles Eberhart, John Hopkins University. The coding sequence of NICD2 was subcloned into pCDH-CMV-EF1-puro through the BamHI and NotI sites. The pLKO-shNotch1 construct was a kind gift of Dr. Adolfo Ferrando, Columbia University. The pLKO-shNotch2 construct was purchased from Open Biosystems (Huntsville, Ala., clone ID, TRCN0000056426). Lentiviruses were produced in 293FT cells as previously described with the packaging plasmids psPAX2 and pCI-VSVG [42]. LY294002, Akt inhibitor III (SH-6), and the γ-secretase inhibitors, DAPT (N-[N-(3,5-difluorophenacetyl)-L-alanyl]-Sphenylglycine t-butyl ester) or L685,458, (5S)-(t-Butoxycarbonylamino)-6-phenyl-(4R)hydroxy-(2R)benzylhexanoyl)-L-leu-L-phe-amide) were purchased from Calbiochem (Gibbstown, N.J.).

Radiation. Cells were irradiated as single cell suspension for CD133+ cells or as monolayer culture for CD133-negative cells using an AGFA X-RAD 320 irradiation system at 0.85 Gy/min.

Growth Curve, caspase 3/7 assay, and clonogenic survival assay. Cells were infected with lentivirus or treated as described in figure legends. Following radiation, cells were aliquoted into 96-well plate at 3000 cells per well in triplicates. Cell number was measured for 5 days using the CellTiter-Glo assay kit (Promega, Madison, Mich.). Averaged cell titer of the control group on day 1 was assigned a value of 1. All other relative cell titers were normalized accordingly.

The caspase activities at 48 or 72 hours after radiation were measured using the Caspase-Glo 3/7 assay (Promega) according to the manufacturer's instructions. The caspase activities were normalized to the corresponding cell titers to obtain the relative caspase 3/7 activities.

To measure clonogenic survival, CD133+ glioma cells were plated in 24-well plates at 50 cells per well for the sham-irradiated groups, 100 cells per well for cells irradiated at 1 Gy, or 500 cells per well for cells irradiated at 2 or 3 Gy. Eight wells were plated for each group. Fourteen days after plating, neurospheres containing more than 50 cells were scored. Alternatively, CD133-negative glioma cells were plated in 6-well tissue culture plates in triplicates at 100 cells per well for the sham-irradiated groups, 200 cells per well for cells irradiated at 1 Gy, or 500 cells per well for cells irradiated at 2 or 3 Gy. Twenty-one days after radiation, cells were fixed and stained with 0.5% crystal violet. Colonies consisting of more than 50 cells were scored. To determine the surviving fractions, the number of neurosphere at each radiation dose was normalized to that of the corresponding unirradiated control group.

Luciferase assay. The Notch-responsive luciferase reporter was purchased from SABiosciences (Frederick, Md.), which was pre-mixed with the internal control construct directing constitutive expression of the Renilla luciferase. Cells were transfected with the luciferase reporter by FuGene 6 (Roche). The luciferase activities were determined by the Dual-luciferase assay system (Promega).

Real-time PCR. Total RNA was prepared using the RNeasy kit (Qiagen, Valencia, Calif.), and reverse transcribed into cDNA by iScript cDNA synthesis kit (BioRad, Hercules, Calif.). Real-time PCR was performed on a Bio-Rad iCycler system using SYBR-Green Mastermix (SABiosciences, Frederick, Md.). PCR products were verified by melting curves. The threshold cycle (C_{T}) values for each gene were normalized to C_{T} of β-Actin and HPRT1 (hypoxanthine phosphoribosyltransferase 1). The primers used were as follows: Notch1, forward 5′-CGC ACA AGG TGT CTT CCA G, reverse 5′-AGG ATC AGT GGC GTC GTG; Notch 2, forward 5′-TGG TGG CAG AAC TGA TCA AC, reverse 5′-CTG CCC AGT GAA GAG CAG AT; Hes4, forward 5′-TGG ACG CCC TCA GAA AAG, reverse 5′-GCT CCG CAG GTG TCT CAC; Hes5, forward 5′-TGG AGA AGG CCG ACA TCC T, reverse 5′-GGC GAC GAA GGC TTT GC.

Xenograft formation assay. T4302 CD 133+ glioma cells lentivirally infected and selected for expression of the puromycin marker were left unirradiated or irradiated at 3 Gy. Immediately after radiation, cells (250,000 cells per injection) were implanted into both flanks of athymic BALB/c nu/nu mice in accordance with a protocol approved by Duke University Institutional Animal Care and Use Committee. Three mice were inoculated for a total of six injections per group. Mice were maintained for 4 weeks until diameters of the largest tumors reached approximately 20 mm, Tumor growth was monitored twice a week.

In addition, the invention is not intended to be limited to the disclosed embodiments of the invention. It should be understood that the foregoing disclosure emphasizes certain specific embodiments of the invention and that all modifications or alternatives equivalent thereto are within the spirit and scope of the invention as set forth in the appended claims.

### REFERENCES

1. DeAngelis L M. Brain tumors. N Engl J Med. 2001; 344:114-123.
2. Grossman S A, Batara J F. Current management of glioblastoma multiforme. Semin Oncol. 2004; 31:635-644.
3. Stupp R, Mason W P, van den Bent M J, et al. Radiotherapy plus concomitant and adjuvant temozolomide for glioblastoma. N Engl J. Med. 2005; 352:987-996.
4. Furnari F B, Fenton T, Bachoo R M, et al. Malignant astrocytic glioma: genetics, biology, and paths to treatment. Genes Dev. 2007; 21:2683-2710.
5. Clarke M F, Dick J E, Dirks P B, et al. Cancer stem cells—perspectives on current status and future directions: AACR Workshop on cancer stem cells. Cancer Res. 2006; 66:9339-9344.
6. Bao S, Wu Q, Sathornsumetee S, et al. Stem cell-like glioma cells promote tumor angiogenesis through vascular endothelial growth factor. Cancer Res. 2006; 66:7843-7848.
7. Bao S, Wu Q, McLendon R E, et al. Glioma stem cells promote radioresistance by preferential activation of the DNA damage response. Nature. 2006; 444:756-760.
8. Singh S K, Hawkins C, Clarke I D, et al. Identification of human brain tumor initiating cells. Nature. 2004; 432:396-401.
9. Galli R, Binda E, Orfanelli U, et al. Isolation and characterization of tumorigenic, stem-like neural precursors from human glioblastoma. Cancer Res. 2004; 64:7011-7021.
10. Singh S K, Clarke I D, Terasaki M, et al. Identification of a cancer stem cell in human brain tumors. Cancer Res. 2003; 63:5821-5828.
11. Hemmati H D, Nakano I, Lazareff J A, et al. Cancerous stem cells can arise from pediatric brain tumors. Proc Natl Acad Sci USA. 2003; 100:15178-15183.
12. Beier D, Hau P, Proescholdt M, et al. CD133(+) and CDI33(−) glioblastoma-derived cancer stem cells show differential growth characteristics and molecular profiles. Cancer Res. 2007; 67:4010-4015.
13. Son M J, Woolard K, Nam D H, et al. SSEA-1 is an enrichment marker for Minor-initiating cells in human glioblastoma. Cell Stem Cell. 2009; 4:440-452.
14. Diehn M, Cho R W, Lobo N A, et al. Association of reactive oxygen species levels and radioresistance in cancer stern cells. Nature. 2009; 458:780-783.
15. Lai E C. Notch signaling: control of cell communication and cell fate. Development. 2004; 131:965-973.
16. Chiba S. Notch signaling in stem cell systems. Stem Cells. 2006; 24:2437-2447.
17. Wu J, Bresnick E H. Bare rudiments of notch signaling: how receptor levels are regulated. Trends Biochem Sci. 2007; 32:477-485.
18. Huppert S S, Le A, Schroeter Ell, et al. Embryonic lethality in mice homozygous for a processing-deficient allele of Notch'. Nature. 2000; 405:966-970.
19. Armogida M, Petit A, Vincent B, et al. Endogenous beta-amyloid production in presenilin-deficient embryonic mouse fibroblasts. Nat Cell Biol 2001; 3:1030-1033.
20. Dontu G, Jackson K W, McNicholas E, et al. Role of Notch signaling in cell-fate determination of human mammary stem/progenitor cells. Breast Cancer Res. 2004; 6:R605-615.
21. Farnie G, Clarke R B. Mammary stem cells and breast cancer—role of Notch signaling. Stem Cell Rev. 2007; 3:169-175.
22. Fre S, Huyghe M, Mourikis P, et al. Notch signals control the fate of immature progenitor cells in the intestine. Nature, 2005; 435:964-968
23. van Es J H, van Gijn M E Riccio O, et al. Notch/gamma-secretase inhibition turns proliferative cells crypts and adenomas into goblet cells. Nature, 2005; 435:959-963.
24. Hitoshi S, Alexson T, Tropepe V, et al. Notch pathway molecules are essential for the maintenance, but not the generation, of mammalian neural stem cells. Genes Dev. 2002; 16:846-858.
25. Hitoshi S, Seaberg R M, Koscik C, et al. Primitive neutral stem cells from the mammalian epiblast differentiate to definitive neutral stem cells under the control of Notch signaling. Genes Dev. 2004; 18:1806-1811.
26. Nakamura Y, Sakakibara S, Miyata T, et al. The bHLH gene hes1 as a repressor of the neuronal commitment of CNS stem cells. J. Neurosci. 2000; 20:283-293.
27. Weng A P, Ferrando A A, Lee W, et al. Activating mutations of NOTCH1 in human T cell acute lymphoblastic leukemia. Science 2004; 306:269-271
28. Pece S, Serresi M. Santolini E, et al. Loss of negative regulation by Numb over Notch is relevant to human breast carcinogenesis. J Cell Biol. 2004; 167:215-221.
29. Politi K, Feirt N, Kitajewski J. Notch in mammary gland development and breast cancer. Semin Cancer Biol. 2004; 14:341-347.
30. Kanamori M, Kawaguchi T, Nigro J M, et al. Contribution of Notch signaling activation to human glioblastoma multiforme, J Neurosurg, 2007; 106:417-427.
31. Bolos V, Blanco, Medina V, et al., Notch signalling cancer stem cells, Clin Transl Oncol., 2009; 11:11-19.
32. Farni G, Clarke R B, Spence K, et al. Novel cell culture technique for primary ductal carcinoma in situ: role of Notch and epidermal growth factor receptor signaling pathways. J. Natl Cancer Inst. 2007; 99:616-627.
33. Gal H, Amariglio N, Trakhtenbrot L, et al. Gene expression profiles of AML derived stem cells; similarity to hematopoietic stem cells. Leukemia. 2006; 20:2147-2154.
34. Zhang X P, Zheng G, Zou L, et al. Notch activation promotes cell proliferation and the formation of neural stem cell-like colonies in the human glioma cells. Mol Cell Biochem. 2008; 307:101-108.
35. Fan X, Matusi W, Khaki L, et al. Notch pathway inhibition depletes stem-like cells and blocks engraftment in embryonal brain tumors. Cancer Res. 2006; 66:7445-7452
36. Bao S, Wu Q, Li Z, et al., Targeting cancer stem cells through L1CAM suppresses glioma growth. Cancer Res. 2008; 68:6043-6048
37. Eyler C E, Foo W C, LaFiura K M, et al. Brain cancer stem cells display preferential sensitivity to AKT inhibition. Stem Cells. 2008; 26:3027-3036.
38. Li Z, Bao S. Wu Q. et al. Hypoxia-inducible factors regulate tumorigenic capacity of glioma stem cells. Cancer Cell 2009; 15:501-513.
39. Shu Q, Wong K K, Su J M, et al. Direct orthotopic transplantation of fresh surgical specimen preserves CD133+ tumor cells in clinically relevant mouse models of medulloblastoma and glioma. Stem Cells. 2008; 26:1414-1424.
40. Wang J, Wang H, Li Z, et al. c-Myc is required for maintenance of glioma cancer stem cells. PLoS ONE 2008; 3:e3769.
41. Wang Z. Li Y. Banerjee S, et al., Emerging role of Notch in stem cells and cancer. Cancer Lett. 2009; 279:8-12.
42. Scharpfenecker M. Kruse J J, Sprong D. et al. Ionizing radiation shifts the PAI-1/ID-1 balance and activates notch signaling in endothelial cells. Int. J Radiat Oncol Biol Phys. 2009; 73:506-513.
43. Coelho D, Holl v, Weltin D, et al. Caspase-3-like activity determines the type of cell death following ionizing radiation in MOLT-4 human leukemia cells. Br J Cancer, 2000; 83:642-649.
44. Essmann F, Engels I H, Totzke G, et al, Apoptosis resistance of MCF-7 breast carcinoma cells to ionizing radiation is independent of p53 and cell cycle control but caused by the lack of caspase-3 and a caffeine-inhibitable event. Cancer Res. 2004; 64:7065-7072.
45. Kirsch D G, Doseff A, Chau B N, et al. Caspase-3-dependent cleavage of Bcl-2 promotes release of cytochrome c. J Biol Chem. 1999; 274:21155-21161.
46. Michelin S, del Rosario Perez M, Dubner D, et al. Increased activity and involvement of caspase-3 in radiation-induced apoptosis in neural cells precursors from developing rat brain. Neurotoxicology. 2004; 25:387-398.
47. Beel A J, Sanders C R. Substrate specificity of gamma-secretase and other intramembrane proteases. Cell Mol Life Sci. 2008; 65:1311-1334.
48. Kastan M B, Bartek J. Cell-cycle checkpoints and cancer. Nature. 2004; 432:316-323.
49. Sancar A, Lindsey-Boltz L A, Unsal-Kacmaz K, et al. Molecular mechanisms of mammalian DNA repair and the DNA damage checkpoints. Annu Rev Biochem. 2004; 73:39-85.
50. Singh N P, McCoy M T, Tice R R, et al. A simple technique for quantitation of low levels of DNA damage in individual cells. Exp Cell Res. 1988; 175:184-191.
51. Palomero T, Sulis M L, Cortina M, et al. Mutational loss of PTEN induces resistance to NOTCH1 inhibition in T-cell leukemia. Nat Med. 2007; 13:1203-1210.
52. Sade H, Krishna S, Sarin A. The anti-apoptotic effect of Notch-1 requires p561ck-dependent, Akt/PKB-mediated signaling in T cells. J Biol Chem. 2004; 279:2937-2944.
53. Oishi K, Kamakura S, Isazawa Y, et al. Notch promotes survival of neural precursor cells via mechanisms distinct from those regulating neurogenesis. Dev Biol. 2004; 276:172-184.
54. Youle R J, Strasser A. The BCL-2 protein family: opposing activities that mediate cell death. Nat Rev Mol Cell Biol. 2008; 9:47-59.
55. Bae J, Leo C P, Hsu S Y, et al. MCL-1S, a splicing variant of the antitipoptotic BCL-2 family member MCL-1, encodes a proapoptotic protein possessing only the BH3 domain. J Biol Chem. 2000; 275:25255-25261.
56. Ban J, Bennani-Baiti I M, Kauer M, et al. EWS-FLI1 suppresses NOTCH-activated p53 in Ewing's sarcoma. Cancer Res. 2008; 68:7100-7109.
57. Purow B W, Sundaresan T K, Burdick M J, et al. Notch-1 regulates transcription of the epidermal growth factor receptor through p53. Carcinogenesis. 2008; 29:918-925.
58. Mungamuri S K, Yang X, Thor A D, et al. Survival signaling by Notch1; mammalian target of rapamycin (mTOR)-dependent inhibition of p53. Cancer Res. 2006; 66:4715-4724.
59. Dotto G P. Notch tumor suppressor function. Oncogene. 2008; 27:5115-5123.
60. Fan X, Mikolaenko I, Elhassan I, et al. Notch1 and notch2 have opposite effects on embryonal brain tumor growth. Cancer Res, 2004; 64:7787-7793.
61. Purow B W, Hague R M, Noel M W, et al. Expression of Notch-I and its ligands, Delta-like-1 and Jagged-1, is critical for glioma cell survival and proliferation. Cancer Res. 2005; 65:2353-2363.
62. Willers H, Dahm-Daphi J, Powell S N. Repair of radiation damage to DNA. Br J. Cancer. 2004; 90:1297-1301.
63. Bolos V, Grego-Bessa J, de la Pompa J L. Notch signaling in development and cancer. Endocr Rev. 2007; 28:339-363.
64. Nicolas M, Wolfer A, Raj K, et al. Notch1 functions as a tumor suppressor in mouse skin. Nat Genet. 2003; 33:416-421.
65. Proweller A, Tu L, Lepore J J, et al. Impaired notch signaling promotes de novo squamous cell carcinoma formation. Cancer Res. 2006; 66:7438-7444.
66. Grimm D, Streetz K L, Jopling C L, et al., Fatality in mice due to oversaturation of cellular microRNA/short hairpin RNA pathways. Nature. 2006; 441:537-541.
67. Perumalsamy L R, Nagala M, Banerjee P, et al. A hierarchical cascade activated by non-canonical Notch signaling and the in TOR-Rictor complex regulates neglect-induced death in mammalian cells. Cell Death Differ. 2009; 16:879-889.
68. Fleisher A S, Raman R, Siemers E R, et al. Phase 2 safety trial targeting amyloid beta production with a gamma-secretase inhibitor in Alzheimer disease. Arch Neurol. 2008; 65:1031-1038
69. Wong G T, Manfra D, Poulet F M, et al. Chronic treatment with the gamma-secretase inhibitor LY-411,575 inhibits beta-amyloid peptide production and alters lymphopoiesis and intestinal cell differentiation. J Biol Chem, 2004; 279:12876-12882.
70. Arbour N, Vanderluit J L, Le Grand J N, et al. Mcl-1 is a key regulator of apoptosis during CNS development and after DNA damage. J. Neurosci. 2008; 28:6068-6078.
71. Wei L H, Kuo M L, Chen C A, et al. The anti-apoptotic role of interleukin-6 in human cervical cancer is mediated by up-regulation of Mcl-1 through a PI 3-K/Akt pathway Oncogene. 2001; 20:5799-5809.
72. Wang J M, Chao J R, Chen W, et al. The antiapoptotic gene mcl-1 is up-regulated by the phosphatidylinositol 3-kinase/Akt signaling pathway through a transcription factor complex containing CREB. Mol Cell Biol. 1999; 19:6195-6206.
73. Hennessy B T, Smith D L, Ram P T, et al. Exploiting the PI3K/AKT pathway for cancer drug discovery. Nat Rev Drug Discov. 2005; 4:988-1004.
74. Hambardzumyan D, Becher O J, Rosenblum M K, et al. PI3K pathway regulates survival of cancer stem cells residing in the perivascular niche following radiation in medulloblastorna in vivo. Genes Dev. 2008; 22:436-448.
75. Imbimbo B P. Alzheimer's disease: [gamma]-secretase inhibitors. Drug Discovery Today: Therapeutic Strategies. 2008; 5:169-175.
76. O'Neil J, Calvo J, McKenna K, et al. Activating Notch1 mutations in mouse models of T-ALL. Blood. 2006; 107:781-785.
77. Rasul S, Balasubramanian R, Filipovie A, et al Inhibition of gamma-secretase induces G2/M arrest and triggers apoptosis in breast cancer cells. Br J Cancer. 2009; 100:1879-1888.
78. Curry C L, Reed L L, Golde T E, et al. Gamma secretase inhibitor blocks Notch activation and induces apoptosis in Kaposi's sarcoma tumor cells. Oncogene. 2005; 24:6333-6344.
79. Hallahan A R, Pritchard J I, Hansen S, et al. The SmoA1 mouse model reveals that notch signaling is critical for the growth and survival of sonic hedgehog-induced medulloblastomas. Cancer Res. 2004; 64:7794-7800.

We claim:

1. A method for inhibiting Notch signaling in a subject, the method comprising:
(a) administering to the subject in need thereof a therapeutically effective amount of a gamma secretase inhibitor (GSI), wherein the GSI inhibits Notch signaling; and
(b) administering a therapeutically effective amount of radiation.

2. A method for treating gliomas in a subject, the method comprising:
(a) administering to the subject in need thereof a therapeutically effective amount of a gamma secretase inhibitor (GSI); and
(b) administering a therapeutically effective amount of radiation.

3. A method for increasing the sensitivity of gliomas to radiation in a subject, wherein the method comprises administering to a subject in need thereof a gamma secretase inhibitor.

4. The method according to claims 1-3, wherein the gamma secretase inhibitor is DAPT, L685,458, or GSI-18.

5. The method according to claims 1-3, wherein the subject has a malignant glioma.

6. The method according to claim 5, wherein the malignant glioma comprise radiation resistant glioma or glioma stem cells.

7. The method according to claims 1-3, wherein the subject is a mammal.

8. The method according to claim 7, wherein the mammal is a human, dog, cat, or mouse.

9. A method for inhibiting Notch signaling in a subject, the method comprising:
(a) administering to the subject in need thereof a therapeutically effective amount of a Notch signaling inhibitor, wherein the inhibitor reduces Notch 1 or Notch 2 signaling or expression; and
(b) administering a therapeutically effective amount of radiation.

10. A method for treating gliomas in a subject, the method comprising:
(a) administering to the subject in need thereof a therapeutically effective amount of a Notch signaling inhibitor; and
(b) administering a therapeutically effective amount of radiation.

11. The method according to claim 9 or 10, wherein the Notch signaling inhibitor is selected from the group consisting of Notch 1 or Notch 2 antisense RNA, shRNA, siRNA, antibody, dominant-negative form of Notch 1 or Notch 2, and combinations thereof.

12. The method according to claim 9 or 10, wherein the subject has a malignant glioma.

13. The method according to claim 12, wherein the malignant glioma comprise radiation resistant glioma or glioma stem cells.

14. The method according to claim 9 or 10, wherein the subject is a mammal.

15. The method according to claim 14, wherein the mammal is a human, dog, cat, or mouse.
